(19) United States
(12) Patent Application Publication
(10) Pub. No.: US 2026/0053487 A1
(43) Pub. Date: Feb. 26, 2026

(54) TISSUE RETRACTION DEVICES, SYSTEMS, AND METHODS

(71) Applicant: Etechnologies, LLC, Las Vegas, NV (US)

(72) Inventor: Matthew Smith, Las Vegas, NV (US)

(73) Assignee: Etechnologies, LLC, Las Vegas, NV (US)

(21) Appl. No.: 19/258,086

(22) Filed: Jul. 2, 2025

Related U.S. Application Data
(60) Provisional application No. 63/667,342, filed on Jul. 3, 2024.

(51) Int. Cl. A61B 17/02

(52) U.S. Cl. CPC A61B 17/025; A61B 17/0206; A61B 2017/0256

(57) ABSTRACT

Systems, devices, and methods for tissue retraction include one or more instruments operatively couplable for retracting and/or retaining tissues or other materials during a procedure. In one aspect, a first instrument includes an elongate rigid body having a longitudinal axis extending from a proximal end to a distal end. The elongate rigid body has an outer polygonal profile and an interior surface defining a round channel extending along the longitudinal axis. The round channel may receive a tubular instrument therethrough. The elongate rigid body includes one or more sets of flanges protruding from lateral sides of the outer polygonal profile. The flanges positively engage one or more respective tracks of a tissue retraction blade. Additional instruments may be provided that operatively couple to the first instrument and/or to one or more tissue retractor blades, for example while the first instrument is coupled to the tissue retractor blades.

### TECHNICAL FIELD

The present disclosure generally relates to the field of orthopedics and spinal surgery, and more particularly, to tissue retractor devices and systems for use in surgery. Related methods are also described.

### BACKGROUND

The spinal column of bones is a highly complex anatomical structure that includes over 20 bones coupled to one another, housing and protecting critical elements of the nervous system having innumerable peripheral nerves and circulatory bodies in close proximity. Despite its complexity, the spine is a highly flexible structure, capable of a high degree of curvature and twist in nearly every direction. The more than twenty discrete bones of an adult human spinal column are anatomically categorized as one of four classifications—cervical, thoracic, lumbar, or sacral—and are coupled together sequentially to one another by a tri-joint complex that consists of an anterior disc and two posterior facet joints. The anterior discs of adjacent bones are cushioned by cartilage spacers referred to as intervertebral discs or vertebrae. The cervical portion of the spine comprises the top of the spine up to the base of the skull and includes the first seven vertebrae. The intermediate 12 bones are thoracic vertebrae and connect to the lower spine comprising the 5 lumbar vertebrae. The base of the spine comprises sacral bones, including the coccyx. With its complex nature, however, there is also an increased likelihood that surgery may be needed to correct one or more spinal pathologies.

Genetic or developmental irregularities, trauma, chronic stress, tumors, and disease can result in spinal pathologies that either limit this range of motion or threaten critical elements of the nervous system housed within the spinal column. In the treatment of diseases, injuries or malformations affecting spinal motion segments, and especially those affecting disc tissue, it has long been known to remove some or all of a degenerated, ruptured or otherwise failing disc. In cases involving intervertebral disc tissue that has been removed or is otherwise absent from a spinal motion segment, corrective measures are taken to ensure the proper spacing of the vertebrae formerly separated by the removed disc tissue.

Corrective measures may include spinal fusion or insertion of a disc prosthesis into the disc space. A variety of systems have been disclosed in the art which achieve immobilization by implanting artificial assemblies in or on the spinal column. Such systems require surgical implantation during a spinal surgery. Such surgical techniques require access to the surgical site through tissue. Invasive techniques may cause tissue necrosis and creep.

During implantation of such systems, retractors and distractors are commonly used. After an incision is made in a patient's skin, a surgeon will normally use a retractor to retract the patient's skin and other tissues (e.g., fat, muscle) to create a surgical opening. A retractor may include a plurality of curved “blades” closed against one another to form a tube prior to insertion. The closed blades of the retractor may be inserted over a guidewire and/or a dilator device which is positioned through the tissue and inserted into the bone at the desired location and/orientation. The surgeon may use a variety of approaches and/or surgical orientations depending on the procedure being performed, the patient's anatomy and condition, and the surgeon's preferences. For instance, a surgeon may approach the spinal column laterally, posteriorly, or anteriorly, among others. Each type of approach has multiple variations. A distractor may then be used to distract two adjacent vertebrae to create space for tissue resection (e.g., removal of deteriorating disk tissue), and immobilization and/or stabilization.

While retractors and associated instruments can significantly reduce trauma and damage to the patient's tissues, there are challenges involved with keeping the retractor device centered over the desired working area and keeping the patient's tissues from creeping underneath the retractor and between the blades and obscuring the surgeon's view of the vertebrae and intervertebral space. Further, navigating the retractor blades down to the surgical site can be challenging, as the patient's tissue may exert forces on the blades causing them to splay or misalign, which can further increase resistance, create further unwanted damage to tissues, and cause other undesirable challenges.

### SUMMARY

The present disclosure describes instruments, devices, systems, kits, and associated methods for tissue retraction that address one or more of the issues identified above. For instance, an instrument includes an elongate body with an outer polygonal profile and an interior round channel. The instrument includes a plurality of flanges configured to engage corresponding slotted tracks in two or more tissue retractor blades to prevent or reduce splaying, shifting, or other undesirable movement of the tissue retractor blades while the instrument is inserted therein. The outer polygonal profile may correspond to an inner space provided in the closed tissue retractor blades, and the inner round channel may be configured to receive one or more dilators and/or a neuromonitoring device therein. The tissue retractor blades may include spaces or reliefs that provide room for one or more other components to be inserted between the tissue retractor blades and the instrument. For instance, the tissue retractor blades may be configured to retain or house a shim in a space between the instrument and the blade. A shim deployment tool may also be inserted into the space to deploy the shim while the tissue retractor blades are closed, and while the instrument is inserted into the closed blades.

According to embodiments of the present disclosure, an instrument for tissue retraction comprises: an elongate rigid body for insertion into a tissue retractor device, the elongate rigid body comprising a longitudinal axis extending from a proximal end to a distal end, the elongate rigid body further comprising: an exterior surface; an interior surface defining a round channel extending along the longitudinal axis, wherein the round channel is configured to receive a tubular instrument therethrough as the instrument is advanced distally over the tubular instrument; a first set of flanges protruding from a first lateral side of the exterior surface, the first set of flanges extending parallel to the longitudinal axis of the elongate body and configured to positively engage a first track in a first blade of the tissue retractor device; and a second set of flanges protruding from a second lateral side of the exterior surface, the second set of flanges extending parallel to the longitudinal axis of the elongate body and configured to positively engage a second track in a second blade of the tissue retractor device.

In some aspects, the exterior surface comprises a first flat surface disposed on the first lateral side of the elongate rigid body and extending along at least a portion of a length of the elongate body. In some aspects, the round channel is open such that the interior surface defines an opening on the second lateral side, and the second set of flanges comprises a first flange and a second flange, and wherein the first flange and the second flange are disposed on opposing sides of the opening. In some aspects, the instrument further comprises a second flat surface orthogonal to the first flat surface, and a third flat surface orthogonal to the first flat surface, wherein the first flat surface, the second flat surface, and the third flat surface provide a rectangular outer profile for at least a portion of the elongate rigid body. In some aspects, the instrument further comprises at least one projection at a proximal portion of the elongate rigid body, the at least one projection comprising a gripping surface. In some aspects, the first set of flanges comprises a first flange and a second flange protruding from the first lateral side of the exterior surface in opposing directions. In some aspects, the second lateral side of the exterior surface is recessed at a distal portion of the elongate rigid body such that a first cross-sectional outer profile of the elongate body at the distal portion is smaller than a second cross-sectional outer profile of the elongate body at a proximal portion of the elongate rigid body. In some aspects, the recess comprises a cutaway of the elongate rigid body for a first length, the cutaway configured to be positioned over a shim coupled to the second blade of the tissue retractor device. In some aspects, the elongate rigid body comprises a tapered distal section that tapers inward toward a distal end of the elongate rigid body. In some aspects, at least one of the first set of flanges or the second set of flanges is discontinuous. In some aspects, the first set of flanges are oriented on a first plane, and the second set of flanges are oriented on a second plane that is parallel to the first plane.

According to another embodiment of the present disclosure, a tissue retractor blade includes: an elongate blade body extending along a longitudinal axis, the elongate blade body comprising: an outer surface having a non-circular elliptical cross-sectional shape; an interior surface defining at least one slotted track extending along the longitudinal axis, wherein the interior surface comprises a plurality of depressions oriented along the longitudinal axes and configured to engage a detent of a shim to retain the shim in place; and a retractor jaw coupling portion comprising an enlarged cross-sectional profile relative to the non-circular elliptical cross-sectional shape of the elongate blade body, wherein the retractor jaw coupling portion is coupled to a proximal portion of the elongate blade body, and wherein the retractor jaw coupling portion comprises a pair of flanges configured to rest in a retractor jaw recess.

In some aspects, the non-circular elliptical cross-sectional shape of the elongate blade body comprises a pair of lateral edges extending along the longitudinal axis, wherein the pair of lateral edges are disposed on a first plane, and wherein a lateral surface of the retractor jaw coupling portion is on a second plane that is obliquely angled relative to the first plane. In some aspects, the at least one slotted track comprises a first slotted track and a second slotted track extending in parallel along the longitudinal axis, the interior surface defines a back surface disposed adjacent to the first slotted track and the second slotted track, and the back surface is recessed such that a channel is defined between the first slotted track and the second slotted track.

According to another embodiment of the present disclosure, an instrument is configured to couple to a tissue retractor system, the instrument comprising: an elongate blade extending along a longitudinal axis; a first rail protruding from a flat surface of the elongate blade; a second rail protruding from the flat surface of the elongate blade, the second rail oriented parallel with the first rail, wherein the first rail and second rail are disposed on either side of a shim deployment channel; a first flange protruding laterally outward from the first rail, and a second flange protruding laterally outward from the second rail, the first and second flanges being configured to positively engage an internal track of the tissue retractor system; and a handle coupled to a proximal end of the elongate blade; wherein the proximal end of the elongate blade defines a shim access opening in communication with the shim deployment channel, wherein the shim access opening and the shim deployment channel are configured to receive a shim deployment tool while the instrument is coupled to the tissue retractor system.

In some aspects, the handle extends orthogonally to the longitudinal axis. In some aspects, the handle extends obliquely to the longitudinal axis. In some aspects, the first rail and the second rail extend along a portion of a length of the elongate blade, wherein the elongate blade comprises a flat section disposed distally of the first rail and the second rail. In some aspects, the first flange extends along a portion of a length of the first rail, and the second flange extends along a portion of a length of the second rail.

In accordance with another embodiment of the present disclosure, a kit includes: a tissue retractor blade, comprising: an elongate blade body extending along a longitudinal axis, the elongate blade body comprising: an outer surface having a non-circular elliptical cross-sectional shape; an interior surface defining at least one slotted track extending along the longitudinal axis, wherein the interior surface comprises a plurality of depressions oriented along the longitudinal axes; and a retractor jaw coupling portion comprising an enlarged outer profile relative to the non-circular elliptical cross-sectional shape of the elongate blade body, wherein the retractor jaw coupling portion is coupled to a proximal portion of the elongate blade body, and wherein the retractor jaw coupling portion comprises a pair of flanges configured to rest in a retractor jaw recess; a first instrument, comprising: an elongate rigid body configured to couple to the tissue retractor blade, the elongate rigid body comprising a longitudinal axis extending from a proximal end to a distal end, the elongate rigid body further comprising: an exterior surface; an interior surface defining a round channel extending along the longitudinal axis; a first set of flanges protruding from a first lateral side of the exterior surface, the first set of flanges extending parallel to the longitudinal axis of the elongate body and configured to positively engage the at least one track in the tissue retractor blade; and a second set of flanges protruding from a second lateral side of the exterior surface, the second set of flanges extending parallel to the longitudinal axis of the elongate body and configured to positively engage a second track in a second tissue retractor blade; and a second instrument configured for insertion into an instrument channel formed between the tissue retractor blade and the first instrument, the second instrument comprising: an elongate body extending along a longitudinal axis; a first rail protruding from the elongate body; a second rail protruding from the elongate body, the second rail oriented parallel with the first rail, wherein the first rail and second rail are disposed on either side of a shim deployment channel; a first flange protruding laterally outward from the first rail, and a second flange protruding laterally outward from the second rail, the first and second flanges being configured to positively engage the at least one track of the tissue retractor blade; and a handle coupled to a proximal end of the elongate body; wherein the proximal end of the elongate blade defines a shim access opening in communication with the shim deployment channel, wherein the shim access opening and the shim deployment channel are configured to receive a shim deployment tool while the second instrument is disposed in the instrument channel.

It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following. One or more features of any embodiment or aspect may be combinable with one or more features of other embodiment or aspect.

### BRIEF DESCRIPTION OF THE DRAWINGS

The accompanying drawings illustrate implementations of the systems, devices, and methods disclosed herein and together with the description, serve to explain the principles of the present disclosure.

FIG. 1 is a photographic view of a tissue retraction system according to an embodiment of the present disclosure.

FIG. 2A is a photographic view of a tissue retraction device in accordance with an embodiment of the present disclosure.

FIG. 2B is a photographic view of a tissue retraction device in accordance with an embodiment of the present disclosure.

FIG. 3 is a top plan view of a tissue retraction device in accordance with an embodiment of the present disclosure.

FIG. 4 is a perspective view of a pair of retractor blades in accordance with an embodiment of the present disclosure.

FIG. 5 is a side elevation view of a retractor blade in accordance with an embodiment of the present disclosure.

FIG. 6A is a top plan view of a pair of retractor blades in accordance with an embodiment of the present disclosure.

FIG. 6B is a bottom plan view of a retractor blade in accordance with an embodiment of the present disclosure.

FIG. 6C is a top plan view of a retractor blade in accordance with an embodiment of the present disclosure.

FIG. 7 is a perspective view of an introducer instrument in accordance with an embodiment of the present disclosure.

FIG. 8 is a perspective view of two sides of an introducer instrument in accordance with an embodiment of the present disclosure.

FIG. 9 is a top plan view of the introducer instrument shown in FIGS. 8 and 9 in accordance with an embodiment of the present disclosure.

FIG. 10A is a bottom plan view of a pair of retractor blades in a closed configuration in accordance with an embodiment of the present disclosure.

FIG. 10B is a perspective view of a distal portion of a tissue retraction system comprising a pair of retractor blades with an introducer instrument inserted therein in accordance with an embodiment of the present disclosure.

FIG. 10C is a bottom plan view of a tissue retraction system comprising a pair of retractor blades with an introducer instrument and a shim inserted therein in accordance with an embodiment of the present disclosure.

FIGS. 11A-11C are perspective views of a tissue retraction subassembly comprising a pair of retractor blades with an introducer instrument inserted therein in accordance with an embodiment of the present disclosure.

FIG. 12A provides two different perspective views of an introducer instrument for a three-blade retractor system in accordance with an embodiment of the present disclosure.

FIGS. 12B-12D are perspective views of a tissue retraction subassembly having three retractor blades in accordance with an embodiment of the present disclosure.

FIGS. 13A-13B are perspective views of a manual retraction device in accordance with an embodiment of the present disclosure.

FIGS. 14A-14C are side elevation views of tissue retraction sub-assemblies including manual retraction devices in accordance with embodiments of the present disclosure period new line

FIG. 15 is a perspective view of a manual retractor instrument inserted into a retraction blade with a shim deployment tool inserted into the manual retractor instrument in accordance with an embodiment of the present disclosure.

FIGS. 16A-16C are side elevation views of a tissue retraction sub-assembly including a manual retraction instrument at various longitudinal positions relative to a retractor blade in accordance with embodiments of the present disclosure.

FIGS. 17A-17B are photographic views of a tissue retraction subassembly including a manual retraction instrument at different longitudinal positions relative to a retractor blade in accordance with embodiments of the present disclosure.

These Figures will be better understood by reference to the following Detailed Description.

### DETAILED DESCRIPTION

For the purpose of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In addition, this disclosure describes some elements or features in detail with respect to one or more implementations or Figures, when those same elements or features appear in subsequent Figures, without such a high level of detail. It is fully contemplated that the features, components, and/or steps described with respect to one or more implementations or Figures may be combined with the features, components, and/or steps described with respect to other implementations or Figures of the present disclosure. For simplicity, in some instances the same or similar reference numbers are used throughout the drawings to refer to the same or like parts. Thus, although a reference number discussed with respect to one figure may not be shown in that figure, the other drawings may be consulted to identify identical, similar, or analogous components that correspond to the reference number.

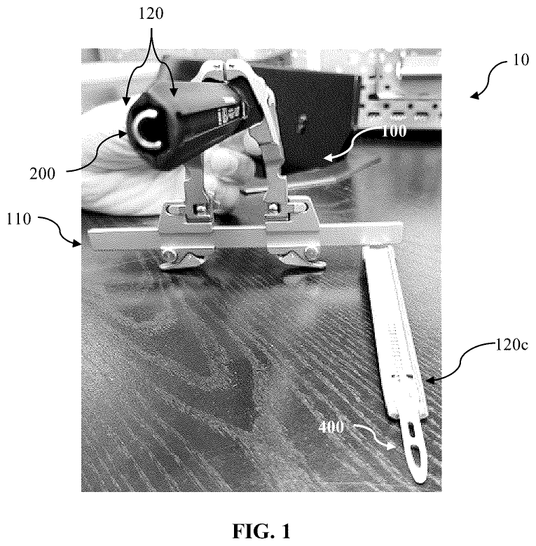

FIG. 1 shows a perspective view of a tissue retraction system 10. The tissue retraction system 10 may be used, for example, in minimally invasive surgical procedures, including orthopedic and/or spinal procedures, to create an open surgical space for the surgeon to perform a procedure while reducing damage to adjacent tissues. The tissue retraction system 10 includes a tissue retractor 100, an introducer 200, and a shim 400. As explained further below, the tissue retraction system 10 may include other components, including a manual retraction assistance instrument, a shim deployment tool, and/or one or more other instruments for use in the spinal procedure.

The tissue retractor 100 is shown in an assembled form, having a set of jaws 130a, 130b operatively coupled to a rail 110. The pair of jaws 130a, 130b may open or closed by sliding relative to one another along the rail 110. In the illustrated embodiment, the jaws 130a, 130b may be connected to the rail 110 by a rack-and-pinion type of engagement. However, it will be understood that other types of connections are also contemplated, including a ratchet mechanism, a friction lock mechanism, a set screw mechanism, and/or any other suitable operative connection. Additionally, the jaws 130a, 130b may be opened by pivoting away from each other by means of a joint through the use of one or more control knobs, as explained further below. Additional modes of movement and adjustment may also be provided to give the surgeon control over the manner and extent of tissue retraction.

A pair of blades 120 are coupled to the jaws 130a, 130b. In the embodiment of FIG. 1, the distal end of the blades 120 is shown. The two blades 120 are closed against one another in a clamshell engagement, forming a tube with an interior space. An introducer instrument 200 is inserted through the tube formed by the pair of blades 120. The engagement and fit of the introducer instrument 200 with respect to the interior surfaces of the blades 120 will be discussed further below. The introducer instrument 200 may positively engage both of the blades 120 to prevent the blades 120 from separating, shifting, or splaying apart as the tissue retractor 100 is advanced down around a guidewire and/or dilator instrument.

In some embodiments, the tissue retractor 100 may include more than two blades 120, such as three, four, five, six, and/or any other suitable number of blades. A third blade 120c is shown in FIG. 1 with a pre-loaded shim 400. The shim 400 is shown in its deployed configuration such that it protrudes distally from the blade 120c. The shim 400 may initially be preloaded such that the distal end of the shim 400 protrudes little if at all from the distal end of the blade 120c. The various subsystems, devices, and components of the system 10 will be described below.

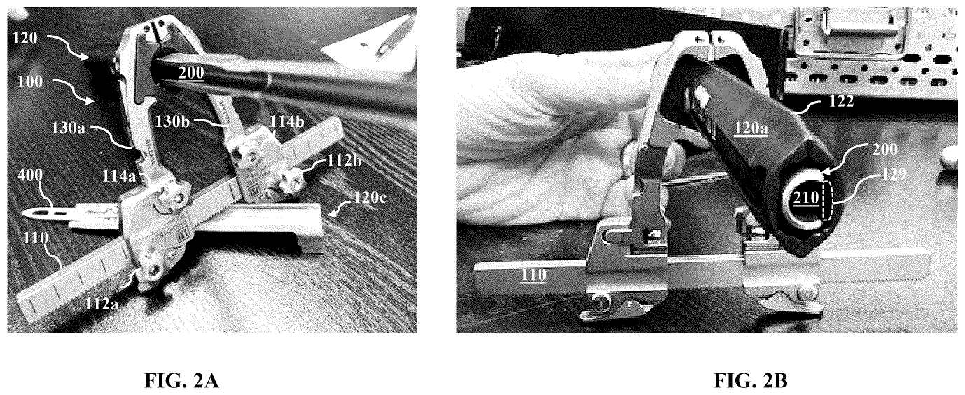

FIG. 2A shows the tissue retractor 100 with the introducer instrument 200 being inserted through a proximal opening in the closed blades 120a, 120b of the tissue retractor 100. As similarly explained above, the tissue retractor 100 includes a pair of jaws 130a, 130b, which are operatively coupled to a rail 110 and are configured to shift laterally relative to one another along the rail 110. The jaws 130a, 130b may be moved manually by the surgeon by grasping the jaws 130a, 130b and manually pulling them apart. Additionally or alternatively, the surgeon may use the knobs or dials 112a, 112b to adjust the lateral positions of the jaws 130a, 130b. The knobs or dials 114 a, 114b may be used to control the toe, splay, or pivotable orientation of the jaws 130a, 130b. Other controls, knobs, dials, and/or any other suitable interface may be provided for other degrees of movement. For example, one or more knobs may be provided to adjust the splay of one or more of the blades 120.

The introducer instrument 200 is shown being inserted through the proximal opening in the closed blades 120. The introducer 200 may be configured to engage a polygonal space 125 formed or defined by the closed blades 120. The introducer 200 may positively engage the blades 120. Coupling features (e.g., flanges) on the introducer 200 engage corresponding features on the interior of the blades 120. This positive engagement keeps the blades 120 in the closed configuration and prevents the blades 120 from opening, shifting, or splaying. At least a portion of the introducer 200 has a rectangular or substantially rectangular outer profile surrounding an open channel configured to receive and be positioned around a dilator or other instrument that has already been inserted into the spine. In this way, the introducer 200 may allow and/or assist to guide the retractor 100 over the dilator or other pre-inserted instrument down to the surgical site (e.g., where the guidewire is inserted into bone or intervertebral tissue). In the illustrated embodiment, the features of the introducer 200 that operatively or positively engage the interior of the blades 120 include flanges or similar projections for engaging slots or channels formed or defined by the interior of the blades 120.

FIG. 2B shows the retractor 100 from a perspective looking toward the distal end of the blades 120. In FIG. 2B, the introducer 200 is inserted through the closed blades 120 such that the distal end of the introducer 200 is visible at the distal opening of the closed blades 120. The closed blades 120 meet at an interface or ridge 122. In the illustrated embodiment, the ridge 122 comprises a spine shape that extends along the length of the closed blades when the introducer 200 is inserted into the closed blades 120. In other embodiments, the interface 122 between the blades 120 may be a non-ridged coupling point. For example, in some embodiments, when the blades 120 are closed, the closed blades 120 may form an elliptical cross-section or an oval cross section. The introducer 200 provides a round channel or opening 210 for receiving a dilator or other pre-inserted instrument. Because the introducer 200 includes an open channel, the distal end of the introducer 200 appears to have a C-shape. In other embodiments, the introducer 200 may be closed such that the distal end forms a circle or other round shape. A space or channel 129 is provided between the distal end of the introducer 200 and the interior surface of the blade 120b. The space 129 may be described as a chamber, a void, a relief, or a channel, and may be configured to provide space or room for another instrument to pass there through even while the introducer 200 is fully inserted into the closed blades 120. In an exemplary embodiment, the space 129 is provided for a preloaded shim 400 such as the shim 400 shown in FIG. 1. Accordingly, the shape of the distal section of the introducer 200 may allow the introducer 200 to be inserted down through the closed blades and over or adjacent to a preloaded shim 400. Further, the coupling between the introducer 200 and the closed blades 120 may provide at least some amount of space 129 through an entire length of the closed blades 120 such that further instruments can be passed down through the space 129 to aid in retracting the blades 120, to deploy the preloaded shim 400, or any other suitable purpose.

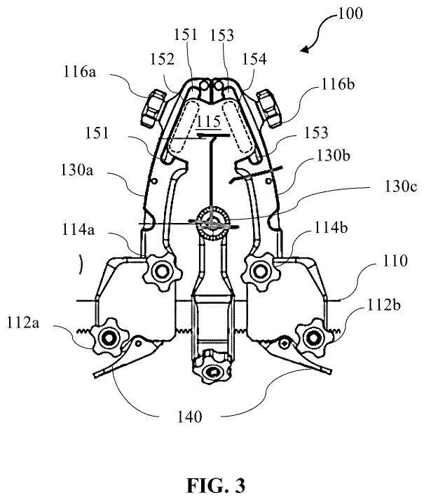

FIG. 3 shows a top view of the retractor 100, according to an embodiment of the present disclosure. The retractor 100 includes a pair of jaws 130a, 130b, which are operatively coupled to the rail 110 as described above. The jaws may be moved and manipulated by the dials 112a, 112b, 114a, 114b as described above. In the illustrated embodiment, an optional third jaw 130c is provided and attached to the rail 110. The third jaw 130c may provide for additional retraction and manipulation of tissue by use of one or more additional knobs. For instance, the jaws 130a and 130b may be configured to pivot and/or slide away from each other to retract tissue in a first direction. The jaw 130c may be configured to pivot or retract in a second direction generally transverse or orthogonal to the first direction for further retraction of the tissue to provide a larger opening for surgical access. However, a person of skill in the art would understand that other relationships and movement profiles of the jaws 130a, 130b, and/or 130b may be implemented or configured without departing from the scope of the present disclosure.

Each of the jaws 130a, 130b includes a respective blade coupling region 152, 154 that receives a respective retractor jaw coupling portion of the retractor blades 120. The jaw 130c may also include a respective retractor jaw coupling portion, in some embodiments. The blade coupling regions 152, 154 are oriented obliquely to one another. In other embodiments, the blade coupling regions 152, 154 may be oriented at a right angle, parallel, or any other suitable orientation. Further each of the jaws 130a, 130b, and/or 130c for a retractor with more than 2 blades comprises a recess 151, 153 to receive and support the retractor jaw coupling portions of the retractor blades 120. Knobs or dials 116a, 116b are provided to adjust the toe of the blades 120, as described above. Levers 140 may be used to unlock and/or lock the locations of the jaws 130a, 130b on the rail 110. In other aspects, the levers 140 may be used to disengage a pawl or teeth from the teeth of the rail 110, allowing the jaws 130a, 130b to slide freely along the track.

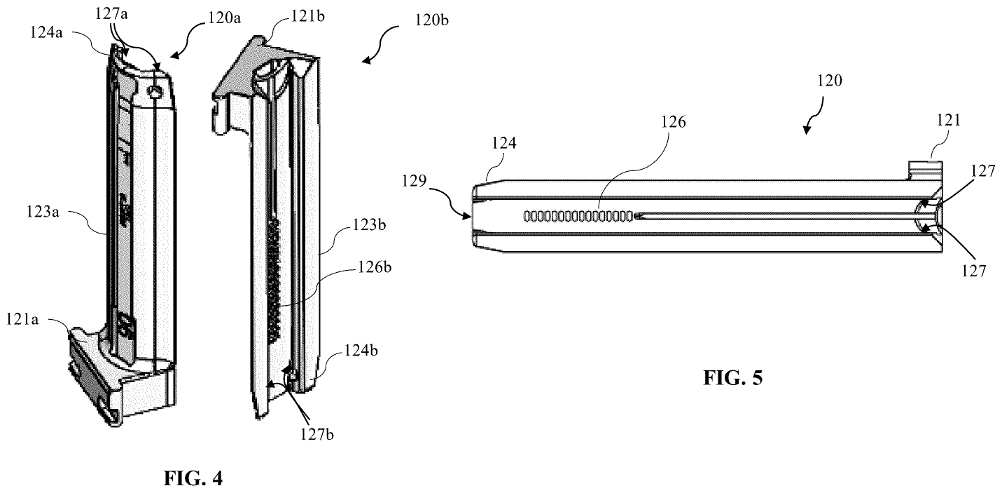

FIG. 4 shows a perspective view of a pair of retractor blades 120a, 120b. Each of the retractor blades 120a, 120b includes a respective retractor jaw coupling portion 121a, 121b. The outer surfaces 123a, 123b of the blades 120a, 120b comprises a non-circular elliptical profile. The non-circular elliptical profile may include a ridged interface 122 between the two blades 120a, 120b, or may be a smooth or non-ridged profile at the interface. Further, each blade 120a, 120b includes a respective distal tapered portion 124a, 124b. The distal tapered portions 124a, 124b may provide an advantageous distal shape to help move tissues out of the way gently as the retractor blades 120 are navigated down to the spine through the tissue.

Further, each of the blades 120a, 120b forms or defines a pair of slots or tracks 127a, 127b configured to receive one or more other instruments as described below. Each track 127a, 127b may comprise a pair of elongate grooves along the longitudinal axes of the blades 120a, 120b. Further, a space or channel may be disposed between each of the tracks 127a, 127b. That space or channel may receive the introducer 200 shown above, a manual retraction device, a shim deployment device, a fiber optic light, or any other suitable instrument. FIG. 5 shows the lateral elevation view of a blade 120. The blade 120 includes the tracks 127 with the space or channel 129 provided there-between. A plurality of notches or depressions 126 are formed in a back surface of the interior of the blade 120. The notches or depressions 126 may be configured to engage a detent or other mechanism of a shim 400 to retain the shim 400 in a preloaded configuration. Each notch 126 may engage or catch a corresponding feature on the shim 400 such that the shim 400 can be advanced to a particular location as desired by the surgeon.

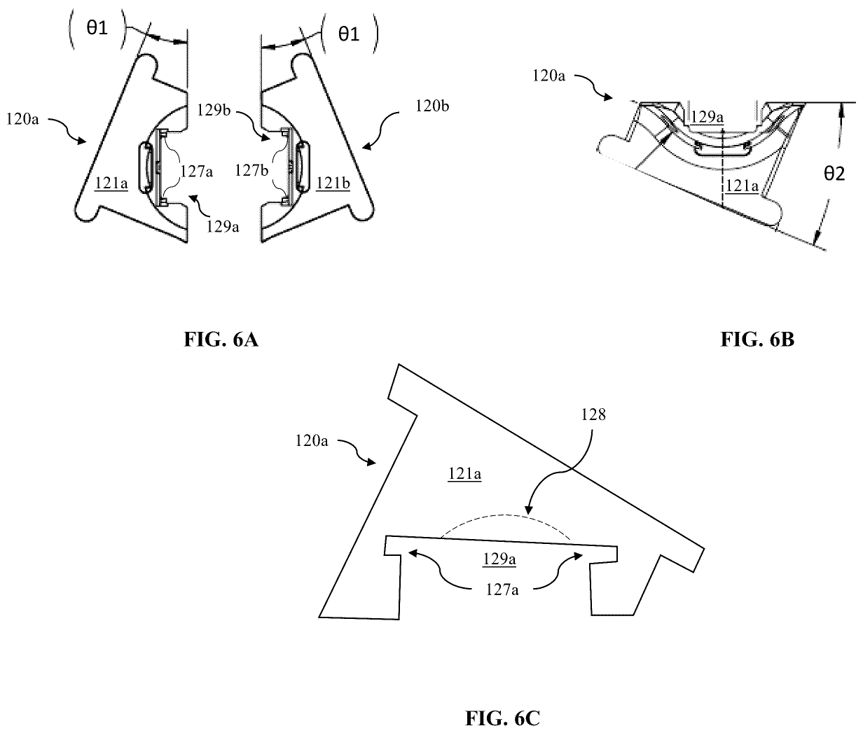

FIGS. 6A and 6B show top views and bottom views, respectively, of the retractor blades 120a, 120b. In FIG. 6A, the retractor jaw coupling portions 121a, 121b are shown from the top or proximal end of the blades 120a, 120b. From this view, the slotted tracks 127a, 127b are shown. The tracks 127a, 127b each include a pair of slots or grooves extending along the longitudinal axis of the blades 120a, 120b. When closed, the blades 120a, 120b provide a rectangular, or substantially rectangular, channel therebetween. Further, between the respective slots are grooves of each track 127a, 127b. The access channels 129a, 129b are provided to allow access for shims, lights, other instruments. The retractor jaw coupling portions 121a, 121b include a back lateral surface that is oriented at an angle θ1 relative to the interfacing surfaces of the blades 120a, 120b. FIG. 6B shows the retractor blade 120a from a distal view such that the clamshell-or shoehorn-shaped outer profile of the blade 120a is shown, as well as the underside of the retractor jaw coupling portion 121a. The deployment channel 129a is also visible from the bottom. The rear side of the retractor jaw coupling portion 121a forms an angle 02 with the interfacing front surface of the blade 120a. in some aspects, θ2 is equal to θ1. In other aspects, 02 may be different from 01.

FIG. 6C shows a top view of a blade 120a according to an embodiment of the present disclosure. In this embodiment, the blade 120a includes a relief or space 128 formed into the back surface of the space 129a. The relief 128 may be accessible from the top surface of the retractor jaw coupling portion 121a by, for example, a shim and shim deployment tool. Accordingly, the relief 128 may provide space for the shim to reside, and for the shim deployment tool to be inserted, while reducing or preventing interference with the introducer instrument described above.

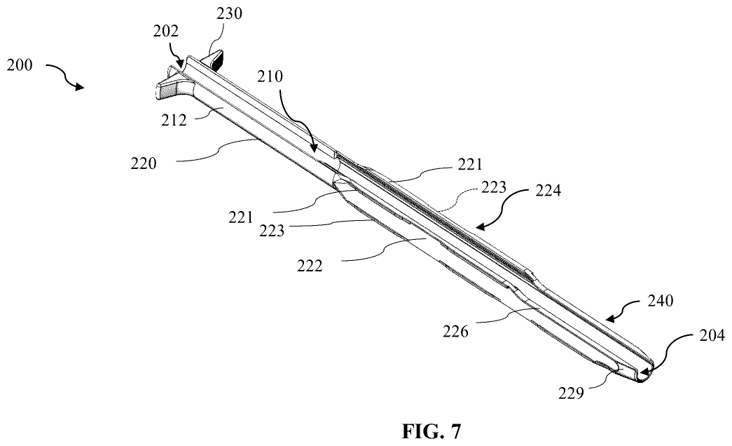

FIG. 7 shows a perspective view of an introducer instrument 200. The introducer element 200 comprises an elongate body 220 comprising an outer surface 212 and an inner surface 214. The inner surface 214 of the elongate body 220 surrounds and defines a round interior channel 210. The round interior channel 210 extends from a proximal opening 202 to a distal opening 204. In some aspects, the round interior channel 210 comprises a cylindrical shape, or a partially cylindrical shape. As shown, the round interior channel 210 is open on one side along an entire length of the elongate body 220. Accordingly, the elongate body 220 may form a half pipe shape. However it will be understood that the body 220 may be more or less than half a cylinder for one or more portions or sections of the elongate body 220.

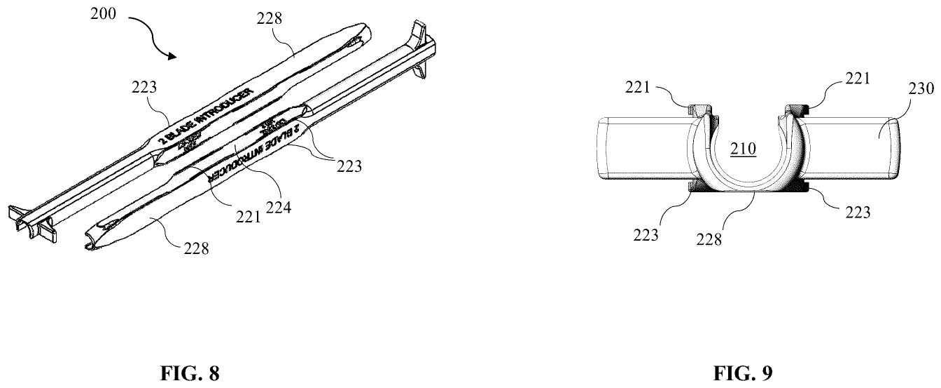

The elongate body 220 comprises at least one polygonal section that includes flat surfaces 222 and 224 (also 228 shown in FIG. 8 below). The polygonal section may be rectangular or substantially rectangular in the outer profile of the body 220. For example, the outer profile or cross-sectional footprint of the body 220 in the rectangular section may be approximately a square. However, the cross-sectional footprint of the body 220 may be any suitable rectangle or non-rectangular polygon. In the illustrated embodiment, the flat surfaces 222, 224 are parallel to each other, and a further flat surface on an underside of the instrument 200 may be orthogonal to the flat surfaces 222, 224. This polygonal profile may correspond to the polygonal space 125 provided by the closed blades 120.

Additionally, a plurality of flanges 221, 223 protrude from the flat surfaces 222, 224, 228. The flanges 221, 223 are shown as discontinuous or fluted. In this way, each flange 221, 223 comprises a plurality of flange sections or portions, each including a leading edge and a trailing edge. The leading edges and trailing edges of each flange 221, 223 transition down to the flat surfaces 222, 224. In some aspects, this configuration may facilitate insertion of the instrument 200 down into the introducer space provided by the closed blades 120. The flanges 221, 223 are configured to positively engage the tracks 127 in the interior surfaces of the blades 120 as explained above. The positive engagement between the flanges 221, 223 and the tracks 127 of each blade 120 prevents the blades 120 from splaying, shifting, or otherwise separating from each other as the tissue retractor 100 is advanced through the tissue down to the surgical site.

A distal portion 226 of the elongate body 220 includes a cutaway or relief 240 that provides space for the shim 400. For instance, the shim 400 may be preloaded into one of the blades 120. The introducer instrument 200 may be advanced through a proximal opening in the closed blades 120 such that the flanges 221, 223 engage the tracks 127 of the closed blades 120. The surgeon may insert the introducer 200 such that the cutaway or relief 240 is aligned with the shim 400 and be positioned over or around the shim 400 when the introducer instrument 200 is fully inserted into the closed blades 120. Accordingly, the introducer instrument 200 does not interfere with or accidentally deploy the shim 400 even when fully inserted. Further, the open channel design of the body 220 may also provide space for other instruments, such as the shim deployment tool, to be inserted into the space 129 between the introducer instrument 200 and the interior surface of the closed blade 120.

At a proximal end of the introducer element, a finger grip 230 is provided to facilitate better grip and manipulation of the introducer instrument 200 by a surgeon or technician. At a distal end portion of the elongate body 220, there is a tapered section 229 that transitions the outer profile of the introducer instrument 200 from a first cross-sectional footprint to a second cross-sectional footprint smaller than the first cross-sectional footprint.

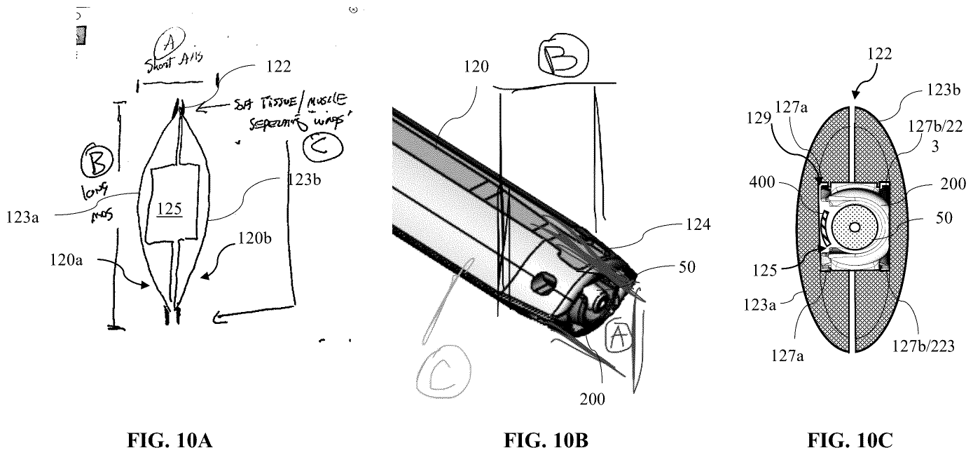

More details regarding the engagement of the flanges 221, 223 with the tracks 127 of the closed blades 120 will be described below with respect to FIG. 10c, for example. The shape and profile of the introducer element, particularly at or around the polygonal section including the flat surfaces 222, 224, may provide several advantages. The outer polygonal profile of the body 220 corresponds to the polygonal space provided in the interior of the closed blades 120. Further, the round interior channel 210 corresponds to the outer profiles and shapes of other instruments, such as dilators and neural stimulation tools, that are typically tubular or cylindrical in shape. Thus, the introducer 200 can be inserted into the closed blades to prevent the blades 120 from splaying or separating while also providing space for the pre-inserted dilators or neuro-monitoring instruments to be received therethrough. Further, the outer profile of the body 220 allows for other instruments to be inserted into the closed blades 120 while the introducer instrument 200 remains inserted into the closed blades. For instance, a shim deployment tool may be inserted there-through to deploy a shim to anchor at least one of the blades 120 to the bone or other tissues. This prevents shifting away from the surgical site. Accordingly, the closed blades 120 can be retained in their desired closed configuration centered around the surgical site before during and after the shim is deployed. When the shim is deployed, the shim deployment tool and the introducer instrument 200 may be removed to provide space for other instruments and to allow the blades 120 to retract the tissue and create additional space for the surgeon to perform the spinal procedure.

FIG. 8 provides two other perspective views of the introducer instrument 200. These views show the rear flat surface 228 of the elongate body 220. The flat surface 224 and the flanges 221, 223 are also shown. FIG. 9 provides a top elevation view of the introducer instrument 200, including the gripping portion 230 and the round interior channel 210. The flanges 221, 223 are also shown. The flanges 221, for example, protrude tangentially from the outer surface of the elongate body 220 and are configured to engage the slotted tracks 127 of the blades 120 as explained above. The introducer instrument 200 may comprise a monolithic or integral body. The introducer instrument 200 may be machined from a single piece of metal. In other embodiments, the introducer instrument 200 may be manufactured by additive manufacturing. In other embodiments, the introducer instrument 200 may be manufactured by welding, bonding, adhering, or otherwise attaching multiple subcomponents together. The flanges 221, 223 are fixed to the body 220 such that their positive engagement with the slotted tracks 127 of the blades 120 retains the closed blades 120 in the closed configuration.

Referring to FIG. 9, the flanges 223 are substantially continuous with the flat surface 228. The flanges 221 are disposed on either side of the open round channel 210. The flanges 221 may protrude tangentially on a plane that is parallel with the plane of the flat surface 228. In other embodiments, the flanges 221, 223 may protrude in other angles or orientations. For instance, the flanges 223 may protrude in opposing oblique directions relative to the plane of the flat surface 228. In some embodiments, the outer profile of the elongate body 220 may be cylindrical or at least partially cylindrical. For example in some embodiments, the elongate body 220 does not include at least one of the flat surfaces 222, 224, 228. In such embodiments, the flanges 221, 223 may extend from the round outer profile of the elongate body 220 a sufficient extent to engage the slotted tracks 127. It will be understood that a variety of configurations and outer profiles are contemplated that include various combinations of the elements and components explained above. For example fewer or more flat surfaces may be provided on the outer surface of the elongate body 220. Further, the flat surfaces 222, 224, 228 may extend for a greater or lesser extent of the length of the introducer instrument 200 than what is illustrated in the Figures above. For example, a majority of the length of the elongate body 220 may include flat surfaces 222, 224, 228. Further, the present disclosure contemplates outer profiles other than cylindrical, elliptical, or rectangular. For example, the outer profile of the polygonal section of the elongate body 220 may be triangular, pentagonal, hexagonal, octagonal, or any other suitable shape. Further, the round interior channel 210 may be closed such that the round interior channel 210 is completely surrounded by an interior surface for at least a portion of the length of the elongate body 220.

FIGS. 10A-10C show various configurations and embodiments of closed blades 120, with or without the introducer instrument 200 inserted therein. FIGS. 10A-10C focus on the distal end or distal portion of the closed blades 120 showing the coupling and physical relationships between the closed blades 120, the introducer instrument 200, and a neuro monitoring instrument 50. FIG. 10A shows the shapes of the outer profiles 123a, 123b of the blades 120a, 120b according to an embodiment of the present disclosure. The outer profiles 123a, 123b comprise a clamshell shape wherein the interface 122 between the individual blades 120a, 120b includes a ridge or spine, which may guide insertion of the closed blades 120a, 120b through tissue (for example, muscle fibers). For example, the physician may insert the closed blades 120a, 120b through a muscle such that the top and bottom ridges 122 of the closed blades 120a, 120b are aligned with the direction of the muscle fibers. This may allow the muscle fibers to stretch and shift allowing the blades to smoothly be inserted through the fibers. The rectangular space 125 is also shown, which may be configured to receive the introducer instrument 200, dilators, neuro-monitoring instruments, fiber optic lights, manual retraction instruments, shim deployment tools, and/or any other suitable instrument as explained herein. Although the diagram shown in FIG. 10A does not show the slotted tracks 127, it will be understood that the slotted tracks 127 may be provided on the interior surfaces that surround the space 125.

FIG. 10B shows the perspective view of the distal portion 124 of the closed blades 120, with the introducer element 200 inserted therein, and a neuro monitoring instrument 50 received within the round channel of the instrument 200. The distal portion 124 of the closed blades 120 may be tapered as explained above to facilitate insertion of the closed blades 120 through the tissue while reducing or minimizing trauma or tissue damage. The outer profiles 123a, 123 b, and the tapered distal portion 124 may also help to retain the blades 120 in their closed configuration.

Referring to FIG. 10C, a distal elevation view is presented of the closed blades 120a, 120b with the introducer instrument 200, a neuro-monitoring instrument 50, and a shim 400 inserted therein. In the embodiment of FIG. 10C, the outer profiles 123a, 123b are oval-shaped or elliptical such that the interface 122 between the blades 120a, 120b comprises a smooth transition. In other words, the interface 122 may not include the ridge shown in FIG. 10A. However, the elliptical profile of the closed blades 120 may also facilitate insertion of the closed blades 120 through tissue while reducing tearing or other trauma as explained above. In other embodiments, the outer profiles 123a, 123b of the blades 120a, 120b may be circular, polygonal, or any other suitable shape.

The introducer instrument 200 is inserted into the space 125 such that the flanges 221, 223 of the introducer instrument engage the slotted tracks 127a, 127b of the blades 120a, 120b. The positive engagement between the flanges 221, 223 and the slotted tracks 127a, 127b prevent the blades 120a, 120b from splaying, shifting, or otherwise separating. The cross-sectional footprint of the introducer instrument 200 occupies a large majority of the cross-sectional footprint of the space 125. In other words, the outermost corners or edges of the introducer instrument 200 located at the outermost corners or edges of the flanges 221, 223, contact, or nearly contact, the respective corners of the space 125, which are located in the slotted tracks 127a, 127b. The neuro-monitoring instrument 50 also occupies all or a great majority of the interior space provided within the round channel 210 of the introducer instrument 200. The relief 240 of the introducer instrument 200 provides space for the preloaded shim 400. Further, there may be sufficient space between the introducer instrument 200 and the interior surfaces of the blade 120a such that a shim deployment tool and/or another instrument may be inserted therein to manipulate the blades 120 and/or to deploy the shim 400.

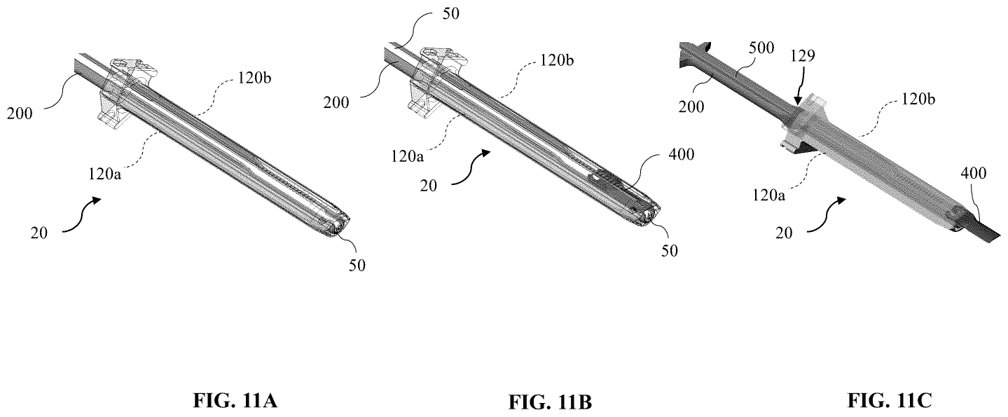

FIGS. 11A-11C illustrate partial retractor assemblies 20 for a shim preloading configuration and shim deployment procedure, according to an embodiment of the present disclosure. It will be understood that the colors of the instruments may not be consistent in FIGS. 11A-11C. For instance, in FIGS. 11A and 11B, the introducer instrument 200 is highlighted in green and the shim 400 is highlighted in blue. The blades 120a, 120b are shown partially transparent as indicated by the dashed lines. In FIG. 11C, the shim 400 is highlighted in red, a shim deployment tool 500 is shown in green, and the introducer instrument 200 is highlighted in blue.

In FIG. 11A, the introducer instrument 200 is inserted into the closed blades 120a, 120b, with the neural monitoring tool 50 shown therein. It will be understood that one or more dilators may be positioned within the round channel of the introducer instrument 200 instead of or in addition to the neuro-monitoring tool 50. In some embodiments, the neuro-monitoring tool 50 is positioned around or incorporated into one or more dilator instruments such that a plurality of tubular instruments are coaxially placed within the round channel 210 of the introducer instrument 200. In FIG. 11A, no shim is preloaded into the relief area 240. A shim 400 may be preloaded into the blade 120b prior to, or during, the tissue retraction procedure. For instance, the shim 400 may be loaded down the slotted tracks of the blade 120b to engage the recesses after the introducer instrument 200 has been inserted into the blades 120a, 120b. In other embodiments, the shim 400 may be loaded into the blade 120b prior to introducing the introducer instrument 200 into the closed blades 120a, 120b.

In FIG. 11B, the shim 400 is shown in the preloaded configuration such that a distal end of the shim 400 does not protrude out the distal opening of the closed blades 120a, 120b. In some embodiments, a surgeon may prefer to preload the shim 400 such that it is positioned more distally or more proximally than the shim 400 as shown in FIG. 11B. For example, the surgeon may prefer to preload the shim 400 such that a small portion of the shim 400 protrudes from the distal opening of the closed blades 120a, 120b.

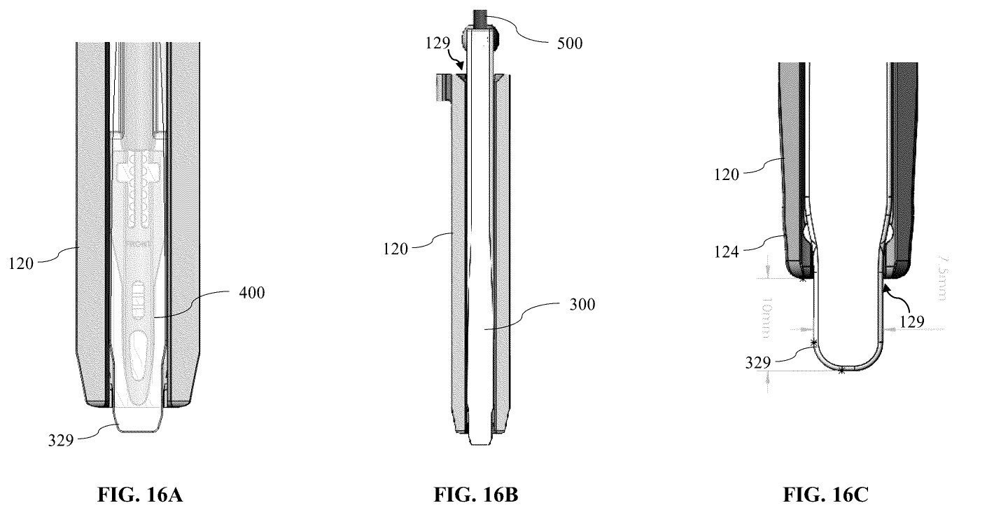

FIG. 11C shows a shim deployment operation. In this embodiment, a shim deployment tool 500 (shown in green) is inserted into a space or channel (129) provided between the introducer instrument 200 and the interior surface of the blade 120b. The shim deployment tool 500 presses on a proximal end of the shim 400 to move the shim through the channel such that the distal tip or end of the shim 400 protrudes distally toward the surgical site. The shim 400 shown in FIG. 11C has a blunt tip which may be desirable for some scenarios. In other embodiments, such as those shown below in FIGS. 16A-16C, the shim 400 may include a pointed tip to penetrate tissues, such as intervertebral disc tissue or bone. In such configurations, the shim 400 may anchor the position of the blade 120b relative to the surgical site.

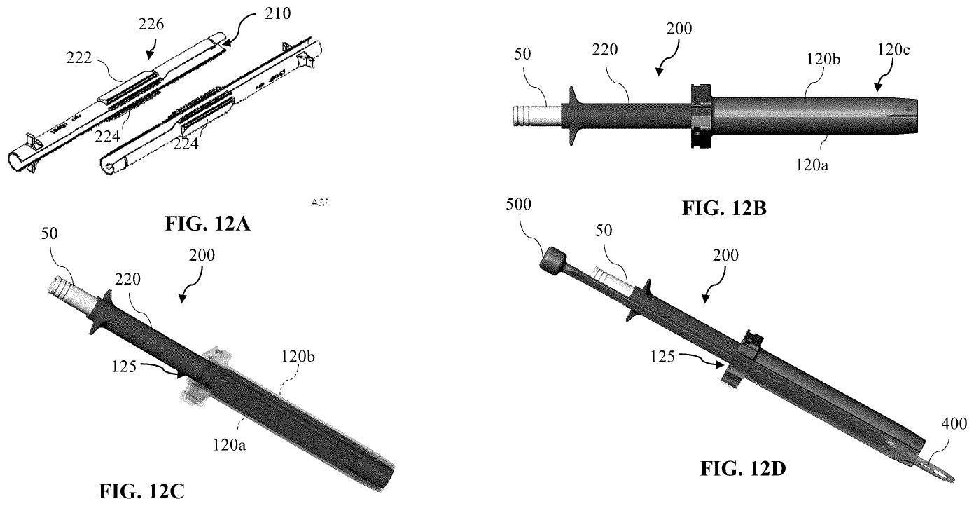

The embodiments of the introducer instrument 200 and the retractor blades 120a, 120b described above are described primarily in the context of two-blade retractor systems. However, the present disclosure also contemplates other configurations with more than two blades. FIGS. 12A-12D illustrate an introducer 200 and blades 120a, 120B, 120C in a three-blade retractor system. In the embodiment shown in FIG. 12A, the introducer instrument 200 includes the flat surfaces 222, 224 that are obliquely angled relative to one another. Similar to the embodiment shown above, flanges may be provided on the introducer instrument 200 to engage slotted tracks in each of the three blades 120a, 120b, 120c. Accordingly, the various flanges extending from the flat surfaces may be grouped and arranged in three planes forming a triangle. Each plane may correspond to one of the blades 120a, 120b, and 120c. The method of engagement with the blades 120 may be similar to that explained above. Each set of flanges engages a respective blade 120a, 120b, 120c to keep the blades in a closed configuration. The outer profile of the introducer instrument 200 includes cylindrical sections and a polygonal section comprising the flat surfaces. However, the introducer instrument 200 may have other designs or arrangements, such as those described above with respect to the two-blade configuration for the introducer instrument 200.

Similar to the embodiments above, a neuro-monitoring instrument 50 is positioned within the round open channel of the introducer instrument 200. In FIG. 12D, a shim deployment tool 500 is inserted into a space between the outer surface of the introducer instrument 200 and the interior surface of the blade 120 to deploy the shim 400.

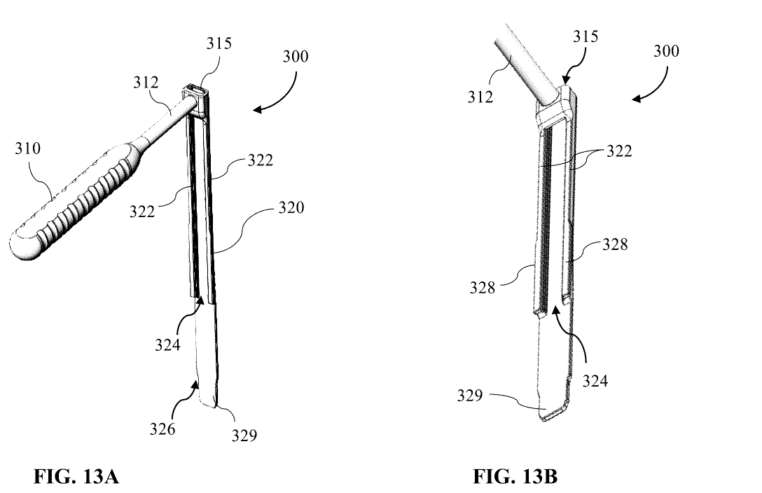

In some aspects, the slotted tracks 127 in the interior surfaces of the blade 120 may be used or occupied by other instruments. For example, some surgeons may prefer to use manual tools to couple to the blades 120 to be able to use greater force to open the blades 120 to provide sufficient space for the operative procedure. In other words, the knobs and dials described above that manipulate the jaws 130 of the tissue retractor 100 may not provide sufficient force or leverage to force the blades open 120 against the resistance of the tissues in the patient. FIGS. 13A-17B illustrate other tools and subassemblies that can be used with the blades 120 to provide similar advantages as those described above and additional advantages.

FIGS. 13A-13B are perspective views of a manual retractor instrument 300. The manual retractor instrument 300 includes a handle 310 coupled to an elongate blade 320. In some aspects, the blade 320 may be referred to as a beam, a flat body, a track engagement portion, or any other suitable term. A pair of rails 322 protrude outward from a front surface of the blade 320 providing a tool space 324 therebetween. Further, a pair of flanges 328 protrude tangentially from the rails 322 in opposing directions. The flanges 328 may be configured to engage the slotted tracks 127 of the blades 120 as similarly explained above. A distal end 329 of the blade 320 may be configured to protrude distally from the closed blades 120. In this way, the distal end 329 may be controlled by the physician to glide against the bone or other tissue to prevent tissue and other material from creeping under the distal ends of the blades 120 which would have otherwise obscured the surgeon's view of the surgical site The handle 310 is coupled to the blade 320 by a neck 312. In some aspects, the handle, neck, blade, rails, and flanges are machined out of a single piece of material. In some embodiments, the manual retractor instrument 300 is monolithic or integral. In other embodiments, the manual retraction instrument 300 may be assembled from multiple components by threaded connections, welds, adhesives, or any other suitable manufacturing method. The neck 312 connects the handle 310 to the blade 320 at a right angle. As explained below, the neck 312 may provide other angulations of the handle 310 with respect to the blade 320, which may provide for other types of leverage or maneuverability. In some embodiments, the neck 312 is resilient and flexible. In other embodiments, the neck 312 is more rigid to reduce flexing when the surgeon provides leverage by the retractor handle 310. For instance, surgeons may use the handles in various ways. The surgeons may pull the handles in opposing directions to pull the respective blades 120 apart. In some aspects, the surgeon may also apply an upward pivoting force on the handle 310 to adjust the toe or splay of the blades at their respective distal ends.

A tool insertion port 315 is also provided at the coupling region between the neck 312 and the blade 320. The tool insertion port 315 comprises a hole or slot in communication with the tool space 324 between the rails 322. In an exemplary embodiment, the surgeon may insert a shim deployment tool through the tool insertion port 315 to deploy a preloaded shim coupled to one of the blades 120. In some aspects, the tool space 324 may be referred to as a tool access channel, a shim deployment channel, or any other suitable term. Similarly, the tool insertion port 315 may be referred to as a tool access port, a shim access opening, or any other suitable term.

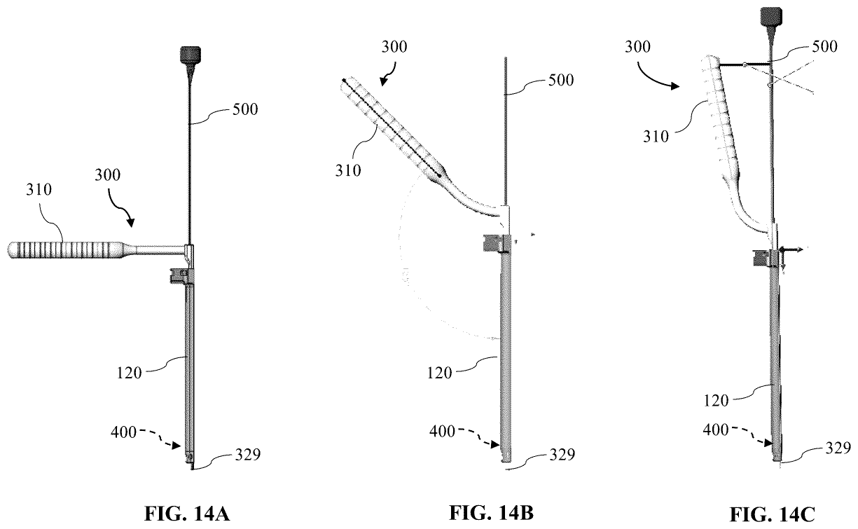

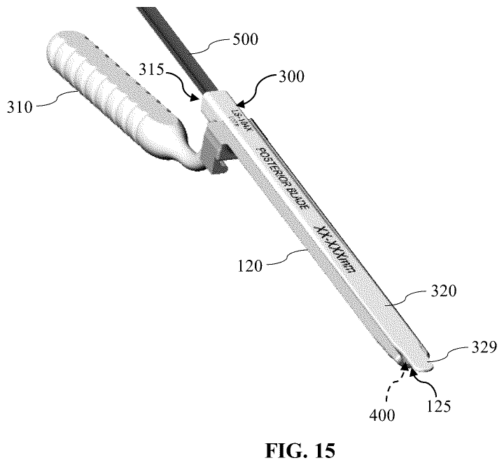

FIGS. 14A-14C illustrate the manual retraction tool 300 attached to a blade 120 with a shim deployment tool 500 being inserted into the tool insertion port consistent with the disclosure above for FIGS. 13A and 13B. In each Figure, a shim 400 is preloaded into a distal portion of the blade 120. The distal end 329 of the manual retraction tool 300 protrudes distally of the blade 120. The handle 310 is shown in three different angular positions or configurations. In some aspects, the three different configurations shown in FIGS. 14A-14C are associated with three different types of manual retraction tools 300. For instance, a manual retraction tool 300 may be provided with any of the angular configurations shown in FIGS. 14A-14C. In other embodiments, the handle 310 may be flexibly attached to the blade of the manual retraction tool 300 such that the handle 310 can flex within the range of motion shown in FIGS. 14A-14C. The shim deployment tool 500 can be inserted through the tool insertion port as explained above through the space 324 to deploy the shim 400.

FIGS. 15, 16A-16C, 17A, and 17B illustrate the manual retraction tool 300 coupled to a blade for shim deployment consistent with the illustrations shown in FIGS. 14A-14C. Referring to FIG. 16A, for example, a partially transparent view of the manual retraction tool 300 shows the preloaded shim 400 positioned behind the distal portion 329 of the manual retraction tool 300. The shim 400 may be disposed adjacent to the relief area 326 described above with respect to FIG. 13A. Non-transparent views are shown in FIGS. 16B and 16C. The manual retraction instrument 300 is inserted into the space 129 such that the flanges 328 of the manual retraction instrument engage the slotted tracks of the blade 120 as similarly explained above. The shim deployment tool 500 can then be inserted through the tool insertion port of the manual retraction instrument 300 to deploy the shim 400.

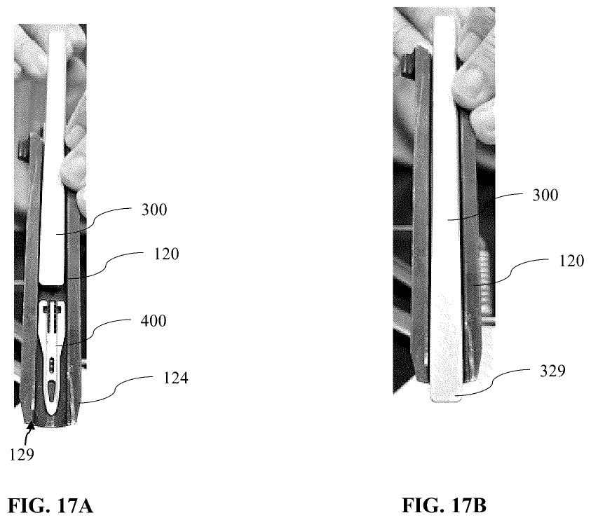

FIGS. 17A and 17B are photographic perspective views of the operative and spatial relationships between the blade 120, the manual retraction instrument 300, and the shim 400. FIG. 17A shows the manual retraction instrument 300 being inserted distally along the tracks into the space 129 with the shim provided in a distal portion of the space 129. FIG. 17B shows the manual retraction instrument 300 advanced distally over the shim 400 such that the distal end 329 protrudes out the distal opening of the blade 120. The flanges of the manual retraction instrument 300 may positively engage the slotted tracks of the blade 120 similar to the introducer 200 to prevent inadvertent removal or decoupling of the manual retraction instrument 300 from the blade 120.

It will be understood that various modifications, combinations, or other variations of the embodiments described above may be made without departing from the scope of the present disclosure. The illustrated embodiments are provided to explain the geometric and functional relationships between various components and their individual features. However, a person of skill in the art would understand that modifications to the shapes, sizes, or arrangement of these components could be made while achieving the same or similar advantages as described above. The components described above may be formed of one or more materials including metallic materials polymeric materials ceramic materials or other types of materials. The components may be formed by additive manufacturing techniques, metal machining, injection molding, metallic stamping, welding, assembly, and/or any suitable combination of manufacturing techniques.

In other words, persons of ordinary skill in the art will appreciate that the implementations encompassed by the present disclosure are not limited to the particular exemplary implementations described above. In that regard, although illustrative implementations have been shown and described, a wide range of modification, change, combination, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An instrument for tissue retraction, comprising:
an elongate rigid body for insertion into a tissue retractor device, the elongate rigid body comprising a longitudinal axis extending from a proximal end to a distal end, the elongate rigid body further comprising:
an exterior surface;
an interior surface defining a round channel extending along the longitudinal axis, wherein the round channel is configured to receive a tubular instrument therethrough as the instrument is advanced distally over the tubular instrument;
a first set of flanges protruding from a first lateral side of the exterior surface, the first set of flanges extending parallel to the longitudinal axis of the elongate body and configured to positively engage a first track in a first blade of the tissue retractor device; and
a second set of flanges protruding from a second lateral side of the exterior surface, the second set of flanges extending parallel to the longitudinal axis of the elongate body and configured to positively engage a second track in a second blade of the tissue retractor device.

2. The instrument of claim 1, wherein the exterior surface comprises a first flat surface disposed on the first lateral side of the elongate rigid body and extending along at least a portion of a length of the elongate body.

3. The instrument of claim 2, wherein the round channel is open such that the interior surface defines an opening on the second lateral side, and wherein the second set of flanges comprises a first flange and a second flange, wherein the first flange and the second flange are disposed on opposing sides of the opening.

4. The instrument of claim 2, further comprising a second flat surface orthogonal to the first flat surface, and a third flat surface orthogonal to the first flat surface, wherein the first flat surface, the second flat surface, and the third flat surface provide a rectangular outer profile for at least a portion of the elongate rigid body.

5. The instrument of claim 1, further comprising at least one projection at a proximal portion of the elongate rigid body, the at least one projection comprising a gripping surface.

6. The instrument of claim 1, wherein the first set of flanges comprises a first flange and a second flange protruding from the first lateral side of the exterior surface in opposing directions.

7. The instrument of claim 1, wherein the second lateral side of the exterior surface is recessed at a distal portion of the elongate rigid body such that a first cross-sectional outer profile of the elongate body at the distal portion is smaller than a second cross-sectional outer profile of the elongate body at a proximal portion of the elongate rigid body.

8. The instrument of claim 7, wherein the recess comprises a cutaway of the elongate rigid body for a first length, the cutaway configured to be positioned over a shim coupled to the second blade of the tissue retractor device.

9. The instrument of claim 1, wherein the elongate rigid body comprises a tapered distal section that tapers inward toward a distal end of the elongate rigid body.

10. The instrument of claim 1, wherein at least one of the first set of flanges or the second set of flanges is discontinuous.

11. The instrument of claim 1, wherein the first set of flanges are oriented on a first plane, and wherein the second set of flanges are oriented on a second plane that is parallel to the first plane.

12. A tissue retractor blade, comprising:
An elongate blade body extending along a longitudinal axis, the elongate blade body comprising:
an outer surface having a non-circular elliptical cross-sectional shape;
an interior surface defining at least one slotted track extending along the longitudinal axis, wherein the interior surface comprises a plurality of depressions oriented along the longitudinal axes and configured to engage a detent of a shim to retain the shim in place; and
a retractor jaw coupling portion comprising an enlarged cross-sectional profile relative to the non-circular elliptical cross-sectional shape of the elongate blade body, wherein the retractor jaw coupling portion is coupled to a proximal portion of the elongate blade body, and wherein the retractor jaw coupling portion comprises a pair of flanges configured to rest in a retractor jaw recess.

13. The tissue retractor blade of claim 12, wherein:
the non-circular elliptical cross-sectional shape of the elongate blade body comprises a pair of lateral edges extending along the longitudinal axis, wherein the pair of lateral edges are disposed on a first plane, and
wherein a lateral surface of the retractor jaw coupling portion is on a second plane that is obliquely angled relative to the first plane.

14. The tissue retractor blade of claim 12, wherein the at least one slotted track comprises a first slotted track and a second slotted track extending in parallel along the longitudinal axis, wherein the interior surface defines a back surface disposed adjacent to the first slotted track and the second slotted track, and wherein the back surface is recessed such that a channel is defined between the first slotted track and the second slotted track.

15. An instrument configured to couple to a tissue retractor system, the instrument comprising:
an elongate blade extending along a longitudinal axis;
a first rail protruding from a flat surface of the elongate blade;
a second rail protruding from the flat surface of the elongate blade, the second rail oriented parallel with the first rail, wherein the first rail and second rail are disposed on either side of a shim deployment channel;
a first flange protruding laterally outward from the first rail, and a second flange protruding laterally outward from the second rail, the first and second flanges being configured to positively engage an internal track of the tissue retractor system; and
a handle coupled to a proximal end of the elongate blade;
wherein the proximal end of the elongate blade defines a shim access opening in communication with the shim deployment channel, wherein the shim access opening and the shim deployment channel are configured to receive a shim deployment tool while the instrument is coupled to the tissue retractor system.

16. The instrument of claim 15, wherein the handle extends orthogonally to the longitudinal axis.

17. The instrument of claim 15, wherein the handle extends obliquely to the longitudinal axis.

18. The instrument of claim 15, wherein the first rail and the second rail extend along a portion of a length of the elongate blade, wherein the elongate blade comprises a flat section disposed distally of the first rail and the second rail.

19. The instrument of claim 15, wherein the first flange extends along a portion of a length of the first rail, and wherein the second flange extends along a portion of a length of the second rail.

20. A kit, comprising:
a tissue retractor blade, comprising:
an elongate blade body extending along a longitudinal axis, the elongate blade body comprising:
an outer surface having a non-circular elliptical cross-sectional shape;
an interior surface defining at least one slotted track extending along the longitudinal axis, wherein the interior surface comprises a plurality of depressions oriented along the longitudinal axes; and
a retractor jaw coupling portion comprising an enlarged outer profile relative to the non-circular elliptical cross-sectional shape of the elongate blade body, wherein the retractor jaw coupling portion is coupled to a proximal portion of the elongate blade body, and wherein the retractor jaw coupling portion comprises a pair of flanges configured to rest in a retractor jaw recess;
a first instrument, comprising:
an elongate rigid body configured to couple to the tissue retractor blade, the elongate rigid body comprising a longitudinal axis extending from a proximal end to a distal end, the elongate rigid body further comprising:
an exterior surface;
an interior surface defining a round channel extending along the longitudinal axis;
a first set of flanges protruding from a first lateral side of the exterior surface, the first set of flanges extending parallel to the longitudinal axis of the elongate body and configured to positively engage the at least one track in the tissue retractor blade; and
a second set of flanges protruding from a second lateral side of the exterior surface, the second set of flanges extending parallel to the longitudinal axis of the elongate body and configured to positively engage a second track in a second tissue retractor blade; and
a second instrument configured for insertion into an instrument channel formed between the tissue retractor blade and the first instrument, the second instrument comprising:
an elongate body extending along a longitudinal axis;
a first rail protruding from the elongate body;
a second rail protruding from the elongate body, the second rail oriented parallel with the first rail, wherein the first rail and second rail are disposed on either side of a shim deployment channel;
a first flange protruding laterally outward from the first rail, and a second flange protruding laterally outward from the second rail, the first and second flanges being configured to positively engage the at least one track of the tissue retractor blade; and
a handle coupled to a proximal end of the elongate body;
wherein the proximal end of the elongate blade defines a shim access opening in communication with the shim deployment channel, wherein the shim access opening and the shim deployment channel are configured to receive a shim deployment tool while the second instrument is disposed in the instrument channel.
